(19) United States
(12) Patent Application Publication
(10) Pub. No.: US 2022/0370735 A1
(43) Pub. Date: Nov. 24, 2022

(54) Aerosol Generation Device with Low Power Mode

(71) Applicant: JT International SA, Geneva (CH)

(72) Inventors: Gordon Montgomery, Ballymena (GB); Shane Day, Warwick (GB)

(73) Assignee: JT International SA, Geneva (CH)

(21) Appl. No.: 17/773,444

(22) PCT Filed: Oct. 29, 2020
(86) PCT No.: PCT/EP2020/080451

§ 371 Date: Apr. 29, 2022

(30) Foreign Application Priority Data
Oct. 30, 2019 (EP) 19206354.3
Oct. 30, 2019 (EP) 19206355.0

(51) Int. Cl. A61M 11/04

(52) U.S. Cl. CPC A61M 11/042; A61M 2205/8212; A61M 2205/3584; A61M 2205/52

(57) ABSTRACT

An aerosol generation device includes an internal clock, a communication interface, and a controller. The controller is configured to record one or more events and apply one or more internal timestamps respectively to the one or more events, the one or more initial timestamps relative to an initial internal time point; receive, by the communication interface, a present external time point; update the internal clock from a present internal time point, relative to the initial internal time point, to the present external time point; and adjust the one or more internal timestamps respectively to one or more external timestamps based upon the difference between the present internal time point and the present external time point.

### FIELD OF INVENTION

The present invention relates to aerosol generation devices, and more specifically low power modes for aerosol generation devices.

### BACKGROUND

Aerosol generation devices such as electronic cigarettes and other aerosol inhalers or vaporisation devices are becoming increasingly popular consumer products.

Heating devices for vaporisation or aerosolisation are known in the art. Such devices typically include a heater arranged to heat a vaporisable product. In operation, the vaporisable product is heated with the heater to vaporise the constituents of the product for the consumer to inhale. In some examples, the product may comprise tobacco; the tobacco may be loose, contained within a capsule, or similar to a traditional cigarette, in other examples the product may be a liquid, or liquid contents in a capsule.

There is a need for improved battery conservation in aerosol generation devices. An object of the invention is, therefore, to address such a challenge.

### SUMMARY

In an aspect there is provided an aerosol generation device arranged to receive a capsule, the aerosol generation device comprising:
a sensor arranged to detect a characteristic of a capsule received in the aerosol generation device; and
a controller configured to:
   detect, by the sensor, that a capsule received in the aerosol generation device is an initiation capsule; and
   initiate a low power state for the aerosol generation device in response to detecting that the initiation capsule has been received in the aerosol generation device.

Preferably, the aerosol generation device is configured to be set to the low power state for shipping and/or storage.

In this way, the aerosol generation device can be set to a low power state for shipping and storage, allowing for the battery of the aerosol generation device to be charged before shipping, with the battery level conserved during shipping and storage for a subsequent first use by a consumer. Moreover, this can be achieved in a standard capsule-based (or cartridge-based) aerosol generation device using existing arrangements, without the need to physically modify the device as the initiation capsule (or cartridge) is inserted in place of a standard capsule containing vaporisable material. This automatic approach to initiating a low power state upon detection of the initiation capsule is quicker and more efficient than manually programming each aerosol generation device to a low power state for shipping and storage.

Preferably, the aerosol generation device arranged to receive an aerosol generating material.

Preferably, the controller is configured to detect, based on the characteristic detected by the sensor, that a capsule received in the aerosol generation device is a low power state initiation capsule.

Preferably, in the low power state, a portion of operating electronics of the aerosol generation device are disabled, or powered off, compared to a normal operating state maintained when the aerosol generation device is in regular use by a consumer.

Preferably the aerosol generation device is arranged to receive a capsule containing a vaporisable substance, such as a fibrous material (e.g. tobacco) or a vaporisable liquid. Preferably the capsule is received in a capsule seating.

Preferably the initiation capsule is a capsule that does not necessarily contain a vaporisable substance, and is instead usable in a production and/or packaging environment place the aerosol generation device into a low power state.

Preferably the initiation capsule has a characteristic that can be sensed by the aerosol generation device to differentiate it from a standard capsule containing a vaporisable substance, such as that used for the generation and inhalation of a vapor by a consumer. This characteristic may be a different capsule size or shape, or instructions stored on an NFC chip in the capsule, amongst others.

Preferably the aerosol generation device is an electronic cigarette.

Preferably the controller is a microcontroller unit comprising one or more processors and memory with instructions stored thereon.

Preferably the controller is configured to disable a portion of operating electronics of the aerosol generation device when initiating the low power state.

In this way, the gradual use of battery power by the operating electronics, during shipping and storage, is minimised.

Preferably the low power state is a power state in which the operating electronics uses less power than in a fully operational power state, a fully operational power state being a power state for vapor generation and inhalation by the consumer.

Preferably disabling a portion of the operating electronics comprises powering off the portion of the operating electronics.

Preferably the controller is configured to disable at least one of a microcontroller unit, a device temperature cut-out sub-circuit, a resistance measurement sub-circuit, a heater driver sub-circuit, a serial flash sub-circuit, or a battery fuel gauge sub-circuit when disabling the portion of operating electronics.

In this way, specific sub-circuits that need not be operational during shipping and storage are powered off to conserve battery charge.

Preferably disabling the device temperature cut-out sub-circuit, the resistance measurement sub-circuit, the heater driver sub-circuit, the linear supply sub-circuit, or the battery fuel gauge sub-circuit comprises powering off the microcontroller unit, the device temperature cut-out sub-circuit, the resistance measurement sub-circuit, the heater driver sub-circuit, the serial flash sub-circuit, or the battery fuel gauge sub-circuit respectively. Preferably powering off the microcontroller unit also powers off the voltage supply to the light emitting diodes.

Preferably the controller is configured to send a trigger to a logic gate array of the operating electronics such that the logic gate array disables the power supply to the portion of the operating electronics to be disabled.

In this way, power can be selectively disabled from specific portions of the operating electronics.

Preferably the controller is further configured to maintain the low power state when the initiation capsule is removed from the aerosol generation device.

In this way, the initiation capsule need not be shipped with the aerosol generation device and can be re-used in the factory environment. This also obviates any confusion on behalf of the consumer as to the purpose of the initiation capsule that they would otherwise have received.

Preferably the aerosol generation device further comprises an indicator, and the controller is further configured to indicate, by the indicator, that the aerosol generation device has entered the low power state.

In this way, it can be determined that the low power state has been successfully entered, thereby ensuring the device is in the low power state for shipping and storage.

Preferably the indicator comprises one or more light emitting diodes.

In this way, a visual indicator is provided that the device has entered the low power state.

Preferably the controller is configured to disable the one or more light emitting diodes to indicate that the aerosol generation device has entered the low power state.

In this way, disabling, or powering off, the light emitting diodes (that would have been switched on as standard when the device is operational) saves power at the battery compared to powering on a separate indicator. This further contributes to the conservation of power for shipping and storage. Moreover, light emitting diodes are typically used as standard in aerosol generation devices; multi-purposing these to indicate entry into the low power state as well as the standard use of conveying information to the consumer obviates the need for further indicators to be incorporated into the aerosol generation device, thereby simplifying manufacturing.

Preferably the aerosol generation device is further arranged to detect a waking trigger condition, and wherein the aerosol generation device is configured to exit the low power state in response to the waking trigger condition.

In this way, when a consumer receives the device, the device can automatically exit the low power state for use by the consumer.

Preferably the waking trigger condition comprises a cable being attached to the aerosol generation device.

In this way, a typical action performed by the consumer, inserting a charging cable, causes the device to exit the low power state. This provides a simple and easily understandable approach for the user to wake the aerosol generation device from the low power state. This improves usability.

Preferably the cable is a charging and/or data cable such as a USB cable. Preferably attaching the cable to the aerosol generation device comprises a connector of the cable being received in a corresponding port of the aerosol generation device. Preferably the second sensor comprises a detector arranged to detect input power and/or input data by the cable.

Preferably the aerosol generation device further comprises an openable cover and the waking trigger condition comprises the openable cover moving between a closed position and an open position.

In this way, a typical action performed by the consumer upon receipt of a new device, opening a cover, causes the device to exit the low power state.

Preferably the openable cover is arranged to cover the capsule seating of the aerosol generation device. Preferably the waking trigger condition comprises detecting that the cover has moved from the closed position to the open position.

Preferably the aerosol generation device further comprises an internal clock, and the controller is configured to set the internal clock to a non-running state when initiating the low power state.

In this way, battery resources are not consumed by running the clock during shipping and storage before a first use by a consumer.

Preferably the controller is further configured to detect and read, by the sensor, the characteristic by a communication chip in a received capsule.

In this way, the controller can determine that the capsule is the initiation capsule and not a standard capsule containing a vapor generating material.

Preferably the controller reads the specific parameter by near field communication.

Preferably the controller is programmed to identify the characteristic as a specific value of a variable field in information stored at the capsule. For example, the variable field can be a ‘Production Date’ field, with the specific value of the production date being set to “00000”.

Preferably the sensor comprises an electrical terminal configured for connection to a corresponding terminal in the initiation capsule, the electrical terminal configured to read information stored in memory in the initiation capsule, and wherein the controller is configured to determine that the information corresponds to the characteristic of the initiation capsule.

In another aspect there is provided an aerosol generation device energy conservation method, the method comprising:
detecting an initiation capsule has been received in the aerosol generation device; and
initiating a low power state for the aerosol generation device in response to detecting that the initiation capsule has been received in the aerosol generation device.

Preferably, the method comprises detecting, based upon a characteristic detected by a sensor, that a low power initiation capsule has been received in the aerosol generation device, wherein the sensor arranged to detect a characteristic of a capsule received in the aerosol generation device.

In another aspect there is provided a non-transitory computer-readable medium storing instructions that, when executed by one or more processors, cause the one or more processors to carry out the steps of:
detecting an initiation capsule has been received in an aerosol generation device; and
initiating a low power state for the aerosol generation device in response to detecting that the initiation capsule has been received in the aerosol generation device.

Preferably, the steps comprise detecting, based upon a characteristic detected by a sensor, that a low power initiation capsule has been received in the aerosol generation device, wherein the sensor arranged to detect a characteristic of a capsule received in the aerosol generation device.

In another aspect there is provided an aerosol generation device comprising:
an internal clock;
a communication interface; and
a controller configured to:
   record one or more events and apply one or more internal timestamps respectively to the one or more events, the one or more initial timestamps relative to an initial internal time point;
   receive, by the communication interface, a present external time point;
   update the internal clock from a present internal time point, relative to the initial internal time point, to the present external time point; and
   adjust the one or more internal timestamps respectively to one or more external timestamps based upon the difference between the present internal time point and the present external time point.

In this way, a consumer can use the aerosol generation device with full timestamp functionality ‘out of the box’ without needing to configure an internal clock of the aerosol generation device. This simplifies the operational setup for the consumer and improves the user experience.

Preferably the internal timestamps are based upon a scale relative to the initial internal time of the aerosol generation device, and the external timestamps are based upon a scale relative to an absolute external time.

Preferably the aerosol generation device is an electronic cigarette.

Preferably the controller is a microcontroller unit comprising one or more processors and memory with instructions stored thereon.

Preferably the controller is further configured to start the internal clock from the initial internal time point in response to determining that the aerosol generation device has exited a low power state.

In this way, a consumer can use a new aerosol generation device, when exiting a low power state configured for shipping and storage, without needing to synchronise or set up the device. Moreover, the low power state allows the aerosol generation device to be provided with a higher battery charge level ‘out of the box’ obviating the need for the consumer to charge the battery of the device before a first use. These advantages combine to improve the overall user experience.

Preferably the low power state is a power state in which operating circuitry of the aerosol generation device uses less power than in a fully operational power state, a fully operational power state being a power state for vapor generation and inhalation by the consumer.

Preferably the trigger comprises detecting that a cable has been attached to the aerosol generation device, or that an openable cover of the aerosol generation device has been moved between a closed and opened position.

Preferably the controller is configured to receive the present external time point, by the communication interface, from an application executed on an electronic device in communication with the aerosol generation device.

In this way, the internal clock of the aerosol generation device can be simply updated using an external time such as that of a smartphone in communication with the aerosol generation device. The consumer does not need to manually configure the internal clock, thereby simplifying the setup of a new aerosol generation device and improving the user experience.

Preferably the controller is configured to update the internal clock to the present external time point when the aerosol generation device first connects to the electronic device.

In this way, the setup ‘out of the box’ of a new aerosol generation device is further simplified by the internal clock being set to the present external time upon the first connection of the aerosol generation device to an electronic device such as a smartphone.

Preferably the present external time point comprises a present clock time of the electronic device.

In this way, the clock time of the electronic device can be used as the clock time of the aerosol generation device, thereby providing a consistency between the devices and improving the interoperability.

Preferably the communication interface is a Bluetooth interface, and the controller is configured to receive the present external time point by a Bluetooth connection to the electronic device using the Bluetooth interface.

In this way, the internal clock of the aerosol generation device can be updated to the external time in a user-friendly manner.

Preferably the controller is configured to update the internal clock by writing the present external time point to the internal clock of the aerosol generation device.

In this way, all timestamps relating to future events can be recorded based upon the external, absolute time.

Preferably wherein the low power state is a power state in which a portion of the operating circuitry used by the aerosol generation device in a fully operational state is disabled.

In this way, power is conserved prior to ‘waking up’ a new aerosol generation device for the first use of the new device by ensuring that non-essential circuitry is not active during shipping and storage.

Preferably a fully operational state is a state in which the aerosol generation device is ready for use by a consumer.

Preferably the internal clock of the aerosol generation device is disabled prior to exiting the low power state.

In this way, power is conserved by not running the internal clock during shipping and storage, prior to ‘waking up’ a new aerosol generation device for the first use of the new device by the consumer.

Preferably when the internal clock is disabled the internal clock is configured to be in a non-running state.

Preferably the low power state is configured for shipping and/or storage of the aerosol generation device.

Preferably the initial internal time point, present internal time point and the one or more internal timestamps are epoch times relative to a reference point internal to the aerosol generation device, and the present external time point and the one or more external timestamps are epoch times relative to a reference point external to the aerosol generation device.

In this way, time adjustments can be efficiently and accurately calculated.

Preferably all epoch times are record in the same format. In an example, the external reference point is an epoch date, such as the Unix reference epoch date 1 Jan. 1970.

Preferably the controller is further configured to determine an activation time point, wherein the activation time point is determined as the difference between the present external time point and the present internal time point.

In this way, a ‘switch on’ time at which the aerosol generation device identifies the trigger condition can be determined on an absolute (external) timescale rather than the relative (internal) timescale. This is beneficial in accurately updating the internal timestamps to external timestamps. This also allows for an associated application on an electronic device to determine if the aerosol generation device has been previously used as, if so, the activation time point will not correspond to the time point at which the aerosol generation device first connected to the electronic device. This improves the quality assurance of the aerosol generation device.

Preferably the controller is configured to adjust a first internal timestamp of the one or more internal timestamps respectively to a first external timestamp of the one or more external timestamps by:
determining a difference between the first internal timestamp and the initial internal time point; and
adding the difference between the first internal time stamp and the initial internal time point to the activation time point.

In this way, the internal timestamps are converted to an external or absolute time. This provides clearer and more user-friendly events for the consumer as the external time is recognisable to the consumer.

Preferably the adjusting process is repeated for each of the internal timestamps of the one or more internal timestamps until all of the internal timestamps are adjusted to respective external timestamps.

Preferably the events comprise data relating to an inhalation on the aerosol generation device.

Preferably the data relating to an inhalation includes at least one of the timestamp, a puff or inhalation duration, a vapor temperature, a fluid or nicotine consumption amount, or a capsule serial code. In this way, information relating to the inhalation that is useful to the consumer can be recorded for the consumer to review.

In another aspect there is provided an aerosol generation device internal clock adjustment method, the method comprising:
recording one or more events and applying one or more internal timestamps respectively to the one or more events, the one or more initial timestamps relative to an initial internal time point;
receiving a present external time point;
updating the internal clock from a present internal time point, relative to the initial internal time point, to the present external time point; and
adjusting the one or more internal timestamps respectively to one or more external timestamps based upon the difference between the present internal time point and the present external time point.

Preferably the method further comprises determining an activation time point, wherein the activation time point is determined as the difference between the present external time point and the present internal time point.

Preferably adjusting a first internal timestamp of the one or more internal timestamps respectively to a first external timestamp of the one or more external timestamps comprises: determining a difference between the first internal timestamp and the initial internal time point; and adding the difference between the first internal time stamp and the initial internal time point to the activation time point.

In another aspect there is provided a non-transitory computer-readable medium storing instructions that, when executed by one or more processors, cause the one or more processors to carry out the steps of:
recording one or more events and applying one or more internal timestamps respectively to the one or more events, the one or more initial timestamps relative to an initial internal time point;
receiving a present external time point;
updating the internal clock from a present internal time point, relative to the initial internal time point, to the present external time point; and
adjusting the one or more internal timestamps respectively to one or more external timestamps based upon the difference between the present internal time point and the present external time point.

Preferably the steps further comprise determining an activation time point, wherein the activation time point is determined as the difference between the present external time point and the present internal time point.

Preferably adjusting a first internal timestamp of the one or more internal timestamps respectively to a first external timestamp of the one or more external timestamps comprises: determining a difference between the first internal timestamp and the initial internal time point; and adding the difference between the first internal time stamp and the initial internal time point to the activation time point.

### BRIEF DESCRIPTION OF THE DRAWINGS

Embodiments of the invention are now described, by way of example, with reference to the drawings, in which:

FIG. 1 is a block diagram of components of an aerosol generation device;

FIG. 2a is a diagram of an example of an aerosol generation device with a closed lid;

FIG. 2b is a diagram of an example of an aerosol generation device with an open lid;

FIGS. 3a, 3b and 3c are diagrams of another example of an aerosol generation device;

FIGS. 3d and 3e are diagrams of another example of an aerosol generation device;

FIG. 3f is a of diagram of a capsule suitable for use with the aerosol generation device of FIGS. 3a-c and 3d-e;

FIG. 3g is a diagram of an electrical terminal arrangement of an aerosol generation device;

FIG. 4 is a block diagram of operating electronics of an aerosol generation device;

FIG. 5 is a block diagram of an aerosol generation device in communication with an external electronic device;

FIG. 6 is a diagram of a graphical user interface of an application associated with an aerosol generation device;

FIG. 7 is a flow diagram of operating steps executed by a controller of an aerosol generation device relating to initiating and exiting a low power mode; and

FIG. 8 is a flow diagram of operating steps executed by a controller of an aerosol generation device relating to a timestamp updating process.

### DETAILED DESCRIPTION

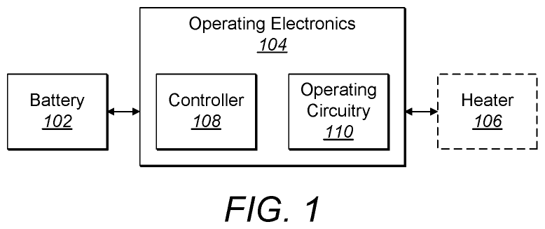

FIG. 1 shows a block diagram of the components of an aerosol generation device (also known as a vapor generation device or electronic cigarette). The aerosol generation device comprises a heater (also referred to as a heater coil) 106, operating electronics or a control device 104, and a battery 102. The battery 102 provides power to the heater 106 and the control device 104. The operating electronics or control device 104 comprises a main control unit (i.e. a controller which can be microcontroller unit (MCU)) 108 and other operating circuitry 110 arranged to control the operation of the aerosol generation device. The controller comprises memory with operating instructions for the aerosol generation device stored thereon, and one or more processors arranged to execute instructions and control the operation of the aerosol generation device.

The heater 106 is arranged to aerosolise or vaporise an aerosol generating material (also known as a vapor generating material). The vapour generating material can be a solid, such as tobacco or a tobacco comprising material; this can be either loose or in a capsule, or in a form similar to a traditional cigarette. The aerosol generating material can also be a liquid, such as a vaporisable liquid stored in a capsule, or any other suitable type of vaporisable material. For the purposes of the present description, it will be understood that the terms vapour and aerosol are interchangeable. In some examples, the heater is arranged within a capsule or cigarette-like aerosol generating material and connectable to the aerosol generation device, rather than being a component of the aerosol generation device itself.

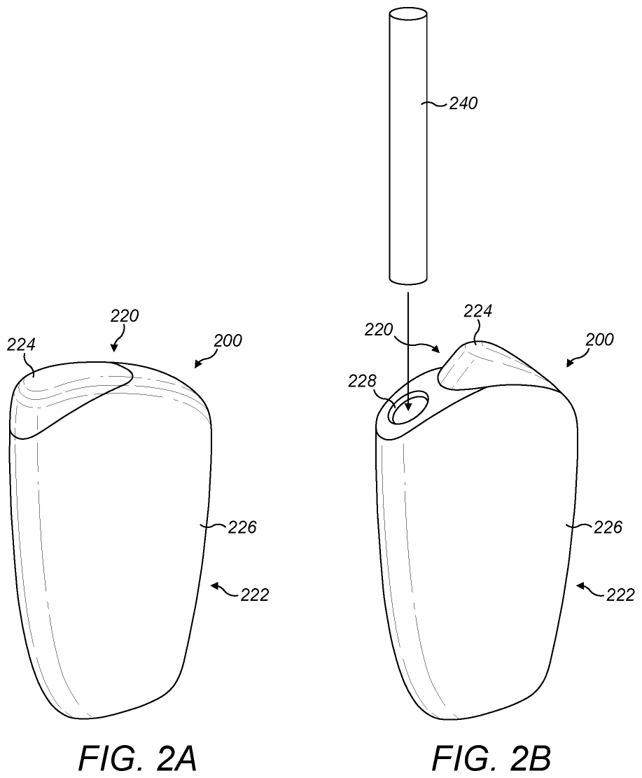

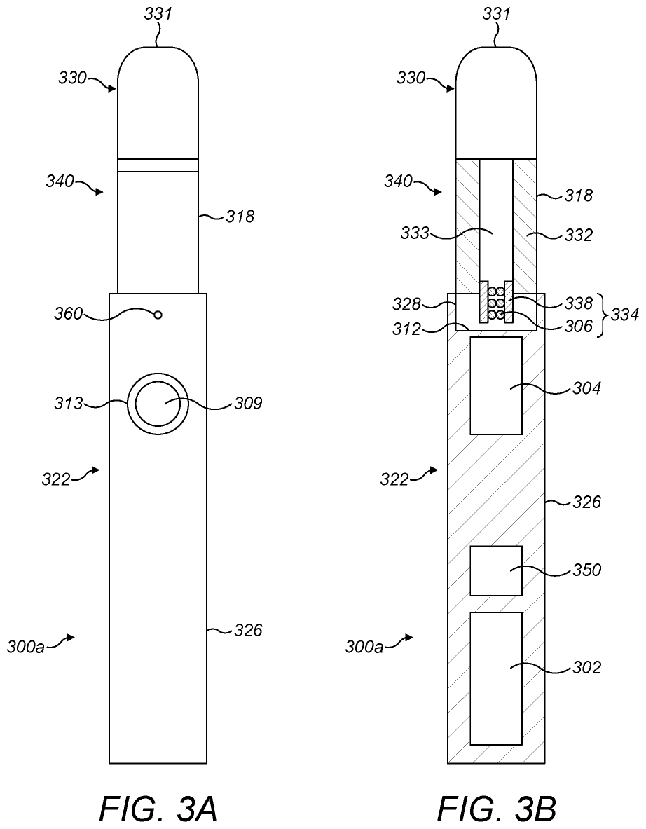

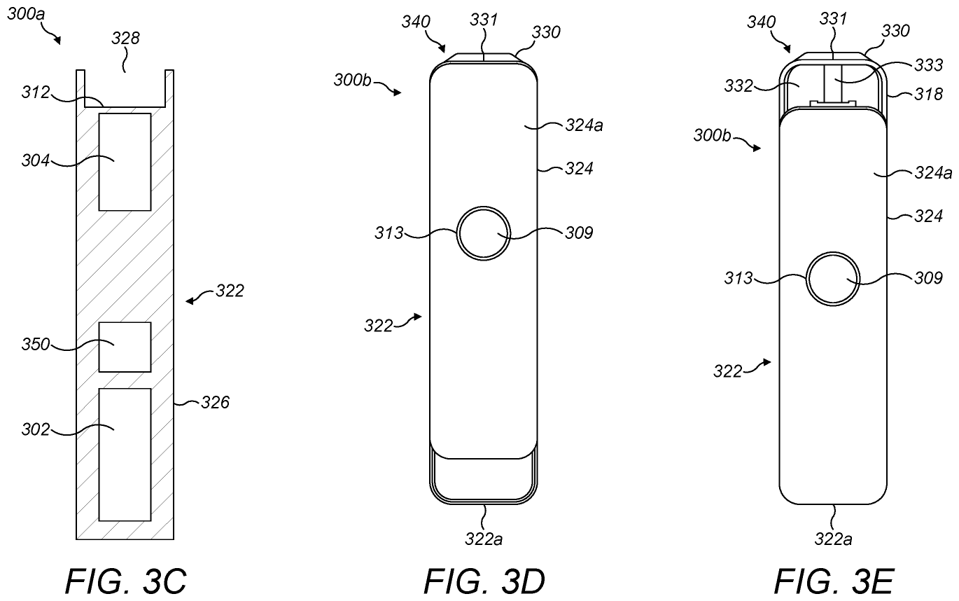

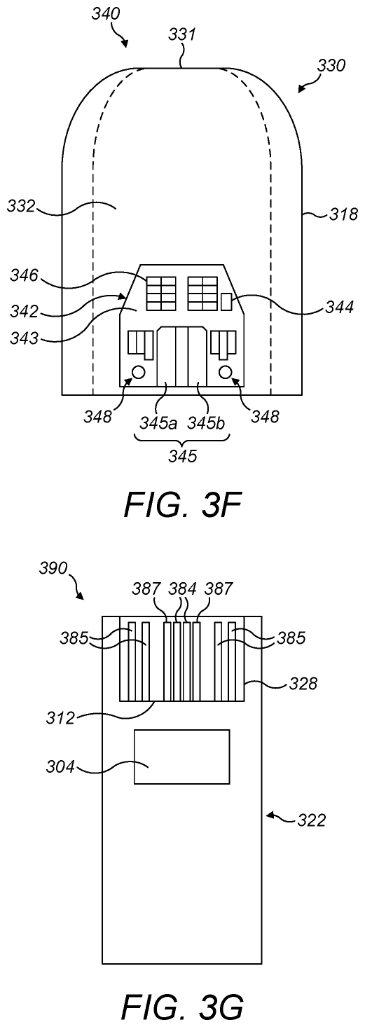

FIGS. 2a to 2b, 3a to 3c, and 3d to 3e show examples of aerosol generation devices in accordance with the block diagram of FIG. 1.

In the example of FIGS. 2a and 2b, the aerosol generation device 200 comprises a main body portion 222 and a lid portion 220. The lid portion 220 includes a lid 224 that is moveably connected to a housing 226 of the main body portion 222.

An opening 228 is arranged in the housing 226; the opening 228 is covered by the lid or cover 224 in a closed position (FIG. 2A) and uncovered (or not covered by the lid 224) in an open position (FIG. 2B).

In an example, the lid 224 is moveably connected to the housing such that it slides between the closed position and the open position. In other terms, the lid 224 is a slideable door moveable between an open and closed position for the opening 228.

Whilst the lid 224 is described as a slideable lid or door in the present description, it will be readily apparent to the skilled person that any other suitable type of lid can be used, such as a hinged lid, a screw-connectable lid, a pop-connectable lid, etc.

The opening 228 is arranged to receive the aerosol generating material. The aerosol generating material 240 can be in a form similar to a traditional cigarette, that is, tobacco wrapped in paper. The cigarette-like aerosol generating material 240 is received in the opening 228, with a distal end of the cigarette-like aerosol generating material 240 extending outwardly from the aerosol generation device so that a consumer can inhale upon it. In alternative arrangements, the aerosol generating material can be contained within a capsule, with the capsule receivable in the opening, or as loose tobacco inserted into the opening.

The heater of the aerosol generation device 200 can be arranged within the housing, in the opening 228, so as to engage the aerosol generating material when received in the opening 228.

The housing further contains the battery 102 and control device 104 including the controller 108 and other operating circuitry 110. A communication interface is further contained within the housing such that the aerosol generation device is communicatively coupleable to an external electronic device, such as a smartphone. In an example, the communication interface is a Bluetooth chip.

FIGS. 3a to 3c show another example of an aerosol generation device 300a. The device of FIGS. 3a to 3c is arranged to receive a capsule 340 containing a aerosol generating liquid, i.e. an aerosol generating capsule 340. FIG. 3f shows a diagram of an aerosol generating capsule 340 suitable for such application.

FIG. 3a shows a diagram of the aerosol generation device 300a with the aerosol generating capsule 340 connected; FIG. 3b shows a cross-sectional diagram of this arrangement. FIG. 3c shows a corresponding cross-sectional diagram with the aerosol generating capsule 340 removed.

The aerosol generation device 300a comprises a main body portion 322 formed by a housing 326. The housing has an opening 328 for receiving the aerosol generating capsule 340. In some examples, a moveable lid (not shown) can also be included to cover the opening which can be operated in substantially the same ways as those described with reference to FIGS. 2a and 2b. In operation, the aerosol generating capsule 340 is received in the opening and connected to a seating 312. The aerosol generating capsule 340 is connected to the seating by a suitable fastening, such as magnetic connection, snap-fit, interference-fit, screw-fit, bayonet-fit, or any other suitable type of connection. In some examples, the capsule contains a heater and the seating is arranged to electronically connect the heater contained within the aerosol generating capsule to the controller and other operating circuitry of the aerosol generation device for the provision of power to the heater. In other examples the heater is within the seating itself and arranged to engage the aerosol generating capsule upon insertion into the opening.

Operating electronics 304, including the controller 108 and other operating circuitry 110, is contained within the housing 326. The housing also contains the communication interface 350, such as a Bluetooth chip, for communicative connection to an external electronic device, and a battery 302 arranged to power the aerosol generation device 300a. A push-button 309 is arranged on an external surface of the housing 326; the push-button is operable to control the aerosol generation device 300a for purposes such as heating the aerosol generating liquid. An indicator, such as a light emitting diode (LED) 313 is also arranged on the external surface of the housing 326; the LED 313 can present indication to the consumer, such as the operational state (i.e. whether the heater is engaged) and a power state of the aerosol generation device 300a. In an example, the LED 313 surrounds the push button 309.

The aerosol generating capsule 340 has a liquid store 332, aerosol channel 333, atomizer arrangement 334 and capsule circuitry (i.e. a capsule chip) 342, housed within a capsule housing 318. The atomizer arrangement 334 includes a heater coil 306 and a wicking material 338. The wicking material 338 is arranged to transfer (or wick) liquid from the liquid store 332 to the heater 306. The heater 306 provides thermal energy to the wicked liquid and generates an aerosol. As an alternative to the liquid and wicking arrangements, the aerosol generating capsule 340 can instead contain a viscous or solid aerosol generating material.

The aerosol generating capsule 340 has a mouthpiece portion 330 with an aerosol outlet mouthpiece opening 331. An aerosol channel 333 is arranged between the mouthpiece opening 331 and the atomizer arrangement 334 such that when a consumer inhales, or draws, on the mouthpiece opening the aerosol generated from the liquid at the heater 306 is drawn through the aerosol channel and out of the mouthpiece opening 331 for inhalation by the consumer. An air inlet 360 may be arranged in the housing 326 of the main body portion 322 or in the aerosol generating capsule 340.

When received in the opening 328, power and data connections are achieved between the aerosol generating capsule 340 and the control device 104 of the main body 322 as described subsequently with reference to FIG. 3f.

FIGS. 3d and 3e show diagrams of another example of an aerosol generation device 300b. The device 300b of FIGS. 3d and 3e is similar to device 300a of FIGS. 3a to 3c, and includes the same features, with the addition of a slideable cover 324.

In the example of FIGS. 3d and 3e, the main body 322 of the aerosol generation device 300b has a slideable cover 324. The slideable cover 324 is arranged to cover the majority of the elongate main body 322 and is slideable in the longitudinal direction of the main body 322 between a first position (FIG. 3d) and a second position (FIG. 3e).

The slideable cover 324 has front 324a and rear panels arranged to cover the major faces of the main body 322.

In the first position (FIG. 3d) the aerosol generating capsule 340 is substantially covered by the slideable cover 324, with the mouthpiece opening 331 exposed so that the user can inhale upon the device. The opposing end 322a of the main body 322 to that which the aerosol generating capsule 340 is fitted is uncovered. In this way, the slideable cover 324 protects the aerosol generating capsule 340. The first position, as shown in FIG. 3d, can be considered as a “closed position” as the aerosol generating capsule 340 is substantially covered by the slideable cover 324.

In the second position (FIG. 3e) the aerosol generating capsule 340 is uncovered; that is the slideable cover 324 has been moved away from the aerosol generating capsule 340, by a sliding action, toward the opposing end 322a of the main body 322. In the second position, considered as an “open position”, the aerosol generating capsule 340 can be inserted/removed from the seating 312.

FIG. 3f shows a cross-sectional diagram of an aerosol generating capsule 340 suitable for use with the aerosol generation devices 300a and 300b of FIGS. 3a to 3c and 3d to 3e. It is to be understood that the dimensions of the aerosol generating capsule 340 are variable; for example the aerosol generating capsule 340 can be more elongate, such as in FIGS. 3a and 3b, to store a larger volume of liquid than that of the more compact capsule in FIG. 3e. The liquid store and aerosol channel of the aerosol generating capsule 340 are not shown in FIG. 3f for clarity; FIG. 3f shows the capsule circuitry 342 which is not shown in FIGS. 3a to 3e, for clarity. Additionally, FIG. 3g shows an electrical terminal arrangement 390 of the seating 312 of the main body 322 of the aerosol generation devices 300a and 300b that is configured for connection to electrical terminals of the aerosol generating capsule 340. The terminals of the capsule circuitry 342 and terminals of the aerosol generation device 300a, 300b combine to provide an interface between the capsule circuitry 342 and the controller 108 of the aerosol generation device 300a, 300b. The terminals in the main body 322 of the aerosol generation devices 300a and 300b can be considered as a sensor or interface for detecting and communication with the aerosol generating capsule 340.

The capsule circuitry 342 comprises electrical terminals including power terminals 345a and 345b, and data terminals 348. The power terminals 345a, 345b are arranged to connect the heater to the battery, via the control device 104 of the aerosol generation device 300a, 300b, by way of corresponding power terminals 384 in the seating 312 of the aerosol generation device 300a, 300b.

The capsule circuitry 342 further comprises memory 344 and a controller 346 for reading/writing from/to the memory 344. The data terminals 348 of the capsule circuitry 342 are arranged to connect to corresponding data terminals 385 in the main body 322 so that the controller 104 in the main body 322 can send and retrieve data from the capsule memory 344. Data stored in the capsule memory 344 can comprise usage data of the aerosol generating capsule 340, authentication data of the aerosol generating capsule 340, the type of aerosol generating capsule 340, a flavour of the material in the aerosol generating capsule 340, a remaining quantity of liquid in the aerosol generating capsule 340, the date of manufacture of the aerosol generating capsule 340, and/or a best before data of the aerosol generating capsule 340, amongst other suitable information. In alternative arrangements, the aerosol generation device 300a, 300b can include a wireless capsule interface with the capsule circuitry 342 of the aerosol generating capsule 340 including a corresponding wireless capsule interface. In this way, the aerosol generation device 300a, 300b can send and retrieve data from the capsule memory 344 by a wireless connection such as near field communication (NFC) or radio-frequency identification (RFID) when the aerosol generating capsule 340 is received in the opening 328. In other alternatives, the aerosol generation device can read capsule information by an optical sensor or image detector.

The terminals 384, 385, 387 of the main body can be configured as elongate conducting members connected at one end to the seating 312, and in turn to the control device 104. The opposing ends of the elongate members form free ends for connection to the corresponding terminals 448 of the aerosol generating capsule 340.

The terminals of the main body 322 can further comprise temperature determination terminals 387. The temperature determination terminals are configured as a measuring circuit that is configured to measure the voltage between the first power terminal 345a and the second power terminal 345b. This voltage can be used for a precise measurement of the heater temperature by determining the resistance of the heater 306.

In an example, the components of the capsule circuitry 342 are arranged on a printed circuit board 343.

With regard to the example aerosol generation devices 200, 300a, 300b in FIGS. 2 and 3, following manufacture of the aerosol generation device 200, 300a, 300b considerable time may elapse before a consumer first uses the device, for example during shipping and storage. From a consumer perspective, it is desirable for the aerosol generation device 200, 300a, 300b to have sufficient battery power for a first use such that it can be used ‘out of the box’ following shipping and storage, without first having to charge the battery. A problem faced in the art is that if considerable time has elapsed during shipping and storage, before the first use, the consumer may find that the aerosol generation device 200, 300a, 300b does not have sufficient battery charge to immediately use the aerosol generation device 200, 300a, 300b. This can be caused by a residual drain on the battery by sub-circuits of the operating electronics. In such a scenario, the consumer will be required to charge the battery before they can use the aerosol generation device 200, 300a, 300b.

To overcome this problem, the aerosol generation device 200, 300a, 300b is placed into a low power mode, by the manufacturer, before being shipped. The aerosol generation device 200, 300a, 300b is then instructed to exit the low power mode upon first use by the consumer. This low power mode preserves battery charge during its shelf life such that the aerosol generation device 200, 300a, 300b will have sufficient battery charge for immediate use by the consumer, ‘out of the box’, without the need to first charge the battery.

In the examples of both FIGS. 2 and 3, the aerosol generation device 200, 300a, 300b is arranged to receive a capsule that initiates the low power mode, i.e. a low power mode initiation capsule. In an example, the initiation capsule is inserted at the end of the manufacturing process before packaging and shipping.

The initiation capsule is inserted into the opening 228, 328 of the aerosol generation device 200, 300a, 300b in a similar way to a cigarette-like aerosol generating material 240 (as in the example of FIG. 2) or in a similar way to a capsule containing an aerosol generating material 340 (as in the example of FIG. 3). The controller uses a sensor arranged in the opening 228, 328 to detect the presence of the capsule and reads capsule information stored at the capsule. From the information the controller determines that the capsule is a low power mode initiation capsule (rather than a standard aerosol generating material containing capsule).

In response to determining that the initiation capsule has been inserted, the controller initiates a low power mode, or low power state, for the aerosol generation device 200, 300a, 300b. The initiation capsule is then removed from the aerosol generation device 200, 300a, 300b so that the aerosol generation device 200, 300a, 300b can be packaged for shipping and sale.

When the initiation capsule is removed from the aerosol generation device, the aerosol generation device 200, 300a, 300b maintains the low power state until a subsequent waking trigger is received. Maintaining the low power state when the initiation capsule is removed is beneficial as the initiation capsule need not be shipped with the aerosol generation device 200, 300a, 300b, and can be re-used in the manufacturing and packaging process of further aerosol generation devices 200, 300a, 300b. This also obviates any confusion on behalf of the eventual consumer as to the purpose of the initiation capsule.

In the case of an aerosol generation device that receives a cigarette-like aerosol generating material 240 (as in FIG. 2), or loose tobacco, the initiation capsule can be suitably dimensioned to be received in the opening 228 into which the cigarette-like aerosol generating material 240 is received.

In the case of an aerosol generation device 300a, 300b that receives capsules containing an aerosol generating material 340 (as in FIG. 3), the initiation capsule can be similarly dimensioned to the standard capsule that contains the aerosol generating material 340 so as to be received in the opening 328 in place of the aerosol generating capsule 340. In an example, the initiation capsule is a dummy capsule that does not contain an aerosol generating material.

In an aerosol generation device 300a, 300b that is arranged to receive aerosol generating material capsules 340 (such as those describe with reference to FIG. 3), the aerosol generation device 300a, 300b can be arranged to read information stored in the memory 344 of the capsule circuitry 342 in such aerosol generating material capsules, as described previously, using an electrical connection or wireless connection. Alternatively the aerosol generation device 300a, 300b can read aerosol generating capsule 340 information using an optical sensor or image detector. The initiation capsule also stores information, for example, by way of a built-in chip. The sensor or interface in the aerosol generation device 300a, 300b is arranged to read this information in the same manners as for an aerosol generating material capsule 340. That is, the sensor or interface is multi-purposed to read information stored in both an aerosol generating material capsule 340 and an initiation capsule. An initiation capsule for use in such an aerosol generation device 300a, 300b can include a modified version of at least one of the parameters stored at a standard aerosol generating material capsule 340; for example the date of manufacture can be set to a specific value, such as 00000, that is indicative of the capsule being an initiation capsule rather than a standard aerosol generating material capsule 340. The controller 108 can determine that the information stored at the initiation capsule is that which triggers the low power mode. The aerosol generation device 300a, 300b is programmed such that the low power mode is initiated on determination of the indicative parameter in the received initiation capsule.

As described, an aerosol generation device 300a, 300b arranged to receive an aerosol generating material capsule 340 has a sensor or interface in the opening 328 used to read information stored at the capsule by, for example, an electrical connection or wireless connection such as an NFC or RFID interface between the aerosol generation device 300a, 300b and the capsule, or by an image detector or optical sensor. The controller can use this sensor to detect and read the initiation capsule in addition to the aerosol generating material capsule 340.

Alternatively, or additionally, a separate dedicated sensor can be arranged in the opening 228, 328 with the specific purpose of detecting the initiation capsule. In particular, such an arrangement can be used in aerosol generation devices 200 that may not otherwise include a capsule sensor or interface, such as aerosol generation devices 200 arranged to receive cigarette-like aerosol generating materials 240 (as described with reference to FIG. 2) or loose tobacco. Also, an aerosol generation device 300a, 300b arranged to receive an aerosol generating material capsule can include such a separate dedicated initiation capsule sensor instead of, or additionally to, the aerosol generating material capsule sensor being multi-purposed to detect the initiation capsule.

In embodiments in which a separate dedicated initiation capsule sensor is used, the initiation capsule parameter can be stored as information which the aerosol generation device 200, 300a, 300b is pre-programmed to recognise as an instruction to enter the low power mode. This need not be a modification of an existing parameter such as the date of manufacture (as devices arranged, for example, to receive cigarette-like aerosol generating materials 240 or loose tobacco may not be compatible with such information); instead it can be a specific parameter for which the sensor is specifically arranged to recognise. That is, the aerosol generation device 200, 300a, 300b can have a sensor specifically arranged to detect an initiation capsule and a low power mode instruction thereon; this sensor need not be a sensor arranged to detect and read aerosol generating material capsules 340. Such a sensor can include an electrical interface in the opening 228, 328 such as that described with reference to FIGS. 3f and 3g between the aerosol generation device 200, 300a, 300b and the initiation capsule. Alternatively, the sensor can include a wireless interface between the aerosol generation device 200, 300a, 300b and the initiation capsule, such as an NFC or RFID interface, wherein the aerosol generation device 200, 300a, 300b can read an NFC or RFID chip in the initiation capsule when the initiation capsule is received in the opening 228, 328. In another alternative, the aerosol generation device 200, 300a, 300b can be arranged to determine that a capsule received in the opening 228, 328 is an initiation capsule by way of an image detector or optical sensor in the opening 228, 328 reading a specific parameter of the initiation capsule.

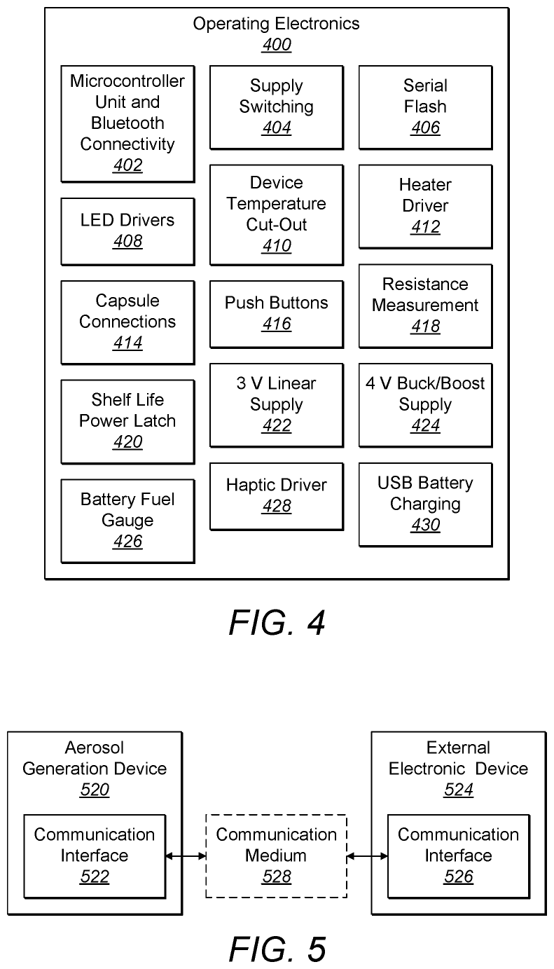

FIG. 4 shows a block diagram of the operating electronics 400 of the aerosol generation device 200, 300a, 300b. The operating electronics 400 of the aerosol generation device comprises a plurality of sub-circuits responsible for operation of various parts of the aerosol generation device. These sub-circuits can include, but are not limited to, a microcontroller unit and Bluetooth connectivity sub-circuit 402, a supply switching sub-circuit 404, a serial flash sub-circuit 406 which uses a memory storage unit to store puff records and event records, a light emitting diode (LED) driver sub-circuit 408, a device temperature cut-out sub-circuit 410, a heater driver sub-circuit 412, a capsule connection sub-circuit 414, a push button sub-circuit 416, a resistance measurement sub-circuit 418, a shelf life power latch sub-circuit 420, a 3 V linear supply sub-circuit 422, a 4 V buck/boost supply sub-circuit 424 used to supply the LEDs with 4 V even when the battery voltage is lower, a battery fuel gauge sub-circuit 426, a haptic driver sub-circuit 428, and a USB battery charging sub-circuit 430, as shown in FIG. 4.

In the low power mode, or low power state, a portion of the operating electronics 400 of the aerosol generation device 200, 300a, 300b are disabled, or powered-off, compared to a normal operating state maintained when the aerosol generation device 200, 300a, 300b is in regular use by the consumer. As such, in the low power state, the operating electronics 400 use less residual power than in a fully operational state.

In more detail, the controller (or MCU) 402 disables specific sub-circuits of the operating electronics 400 (104, 304) when entering the low power mode. The MCU recognises initiation capsule and executes a routine preparing the MCU for power-off. The MCU then triggers a logic gate array to disable a 3 V linear supply sub-circuit 426. This turns off sub-circuits including the device temperature cut-out 410, resistance measurement 418, heater driver 412, battery fuel gauge 426, serial flash 406, and the MCU 402 itself. In turn, turning off the MCU also turns off a 4 V supply to the LED drivers, thereby also turning the LED drivers 408 off.

In more detail, when the controller identifies that the initiation capsule has been received in the aerosol generation device, the output of the shelf-life power latch sub-circuit 420 is turned off. This in turn turns off the output of the 3 V linear supply sub-circuit 422, turning off the supply to the MCU 402 and the output of the supply switching sub-circuit 404. The output of the supply switching sub-circuit 404 supplies sub-circuits including the device temperature cut-out 410, resistance measurement 418 and heater driver 412; these sub-circuits are therefore turned off by turning off the output of the supply switching sub-circuit 404. The output of the 3 V linear supply sub-circuit 422 supplies sub-circuits including the MCU 402, battery fuel gauge 426 and serial flash 406; these sub-circuits are therefore turned off by turning off the output of the 3 V linear supply sub-circuit 422. Any sub-circuits powered by the output of the 3 V linear supply sub-circuit 422 or the output of the supply switching sub-circuit 404 are turned off. As a result of the MCU 402 turning off, the 4 V supply for the LEDs, i.e. the LED driver sub-circuit 408, will also be turned off.

Switching off the MCU also results in the switching off, or suspension, of an internal clock of the aerosol generation device.

The aerosol generation device is provided with an indicator arranged to indicate that the low power mode has been entered, and that the initiation capsule can be removed as the low power mode has been entered. In an example, the indicator is a visual indicator such as one or more LEDS which, when switched off, indicate that the low power mode has been entered. The LEDs are switched off as a consequence of the powering-off of the LED driver sub-circuit 408, as described with reference to FIG. 3.

The indicator allows the manufacturer to know that the aerosol generation device has entered the low power mode, for shipping and storage, and that the initiation capsule can be removed.

Disabling, or powering off, the LED(s) saves power at the battery compared to powering on a separate indicator. This further contributes to the conservation of power for shipping and storage. Moreover, LEDs are typically used as standard in aerosol generation devices; multi-purposing these to indicate entry into the low power state as well as the standard use of conveying information to the consumer obviates the need for further indicators to be incorporated into the aerosol generation device, thereby simplifying manufacturing.

The aerosol generation device 200, 300a, 300b is configured to exit the low power mode in response to a waking trigger condition. This is intended to occur when a consumer first uses a new aerosol generation device, after it has been entered into a low power mode for shipping and storage. That is, the waking trigger is used to instruct a new, ‘out of the box’, aerosol generation device that has not been previously used by a consumer between shipping/storage and this first use, to exit the low power mode. The waking trigger reinstates power to the MCU, and powers on the disabled sub-circuits.

In a first example, the movement of the lid or cover 224, 324 between the closed position (FIG. 2a) and the open position (FIG. 2b) acts as the trigger. An electrical connection can be established when the lid or cover 224, 324 is in the closed position (or the open position) and disconnected when the lid or cover 224, 324 is in the open position (or the closed position). That is, by detection of an electrical connection being connected or disconnected when the lid or cover 224, 324 moves between the two positions, the controller can determine whether the lid or cover 224, 324 is in an open or closed state, and when it is moved between the open and closed states. When the initiation capsule is removed and the aerosol generation device is entered into the low power state, the manufacturer can close the lid or cover 224, 324; the subsequent opening of the lid or cover 224, 324 by the consumer, for example to insert an aerosol generating material, causes power to be reinstated to the MCU, and the aerosol generation device exits the low power mode. Rather than sliding the lid or cover open, opening the lid or cover can also comprise disconnecting a section of the aerosol generation device so as to expose a cavity into which an aerosol generating material capsule can be received, such as disconnecting a mouthpiece portion of the aerosol generation device from a battery portion, with an appropriate switch to detect that something has been removed.

In a second example a waking trigger, that can be used alternatively or additionally to the first waking trigger, can be the detection of a cable having been attached to the aerosol generation device. For example, the cable may be a charging and/or data cable such as USB cable (or any other suitable type of cable, such as a micro-USB, USB-B, USB-C, Lightning cable etc.), receivable in a corresponding port in the aerosol generation device. That is, is the insertion of a cable into a cable port in the aerosol generation device causes power to be reinstated to the MCU and the aerosol generation device to exit the low power mode.

In more detail, the opening of the lid or cover 224, 324, and/or insertion of a cable, switches on the output of the shelf-life power latch sub-circuit 420. In turn this switches on the 3 V linear supply sub-circuit 422. Switching on the 3 V linear supply sub-circuit 422 switches on the MCU 402, battery fuel gauge 426 and serial flash 406 sub-circuits. Switching on the output of the 3 V linear supply sub-circuit 422 also switches on the output of the supply switching sub-circuit 404, and consequently switches on the sub-circuits powered by the supply switching sub-circuit including the device temperature cut-out 410, resistance measurement 418 and heater driver 412 sub-circuits.

In this way, typical actions performed by the consumer, such as inserting a cable or opening a lid or cover 224, 324 causes the aerosol generation device to exit the low power mode. This provides a simple and easily understandable approach for the user to wake the aerosol generation device from the low power state, thereby improving usability.

When the consumer uses the aerosol generation device 520, for each inhalation or puff of generated aerosol or vapor, event data is recorded with a timestamp. Event data can comprise the puff duration, an aerosol or vapor temperature, a fluid and/or nicotine consumption amount, energy consumed per puff, and a capsule serial code amongst others, as well as the timestamp itself. In an example, the fluid and hence nicotine consumption can be calculated based upon the energy consumed per puff, knowing the liquid composition. In another example, the energy consumed per puff can be used to derive information about airflow, and this may be particularly helpful for situations when there is no puff sensor or pressure sensor on the aerosol generation device. As such, using energy consumed per puff as event data is advantageous to provide more information by storing one type of event data. The event data can also include starting and ending points of a puff, puff duration (i.e. the length of a puff) and a puff interval (i.e. the time between consecutive puffs). The event data can also include any further suitable metrics for analysing the behaviour of the consumer. The aerosol generation device 520 is communicatively coupleable to an external electronic device 524, such as a smartphone, as shown in FIG. 5. The aerosol generation device 520 has a communication interface 522 by which it is coupleable to the external electronic device 524 by way of a communication medium between the communication interface 522 of the aerosol generation device 520 and a corresponding communication interface 526 of the external electronic device 524. For example, the communication medium 526 can be a wired connection such as a USB connection, or a wireless connection such as a Bluetooth connection. An application, associated with the aerosol generation device 520, can be loaded on the external electronic device 524. This application can be used to carry out actions including reviewing an aerosol generation device 520 vaping history, or providing instructions to the aerosol generation device 520 via the communication interface 522.

The timestamped event information can be transferred, by the communication interface 522, to the external electronic device 524. This allows the consumer to review their vaping record using a graphical user interface of the associated application provided on a screen of the external electronic device 524.

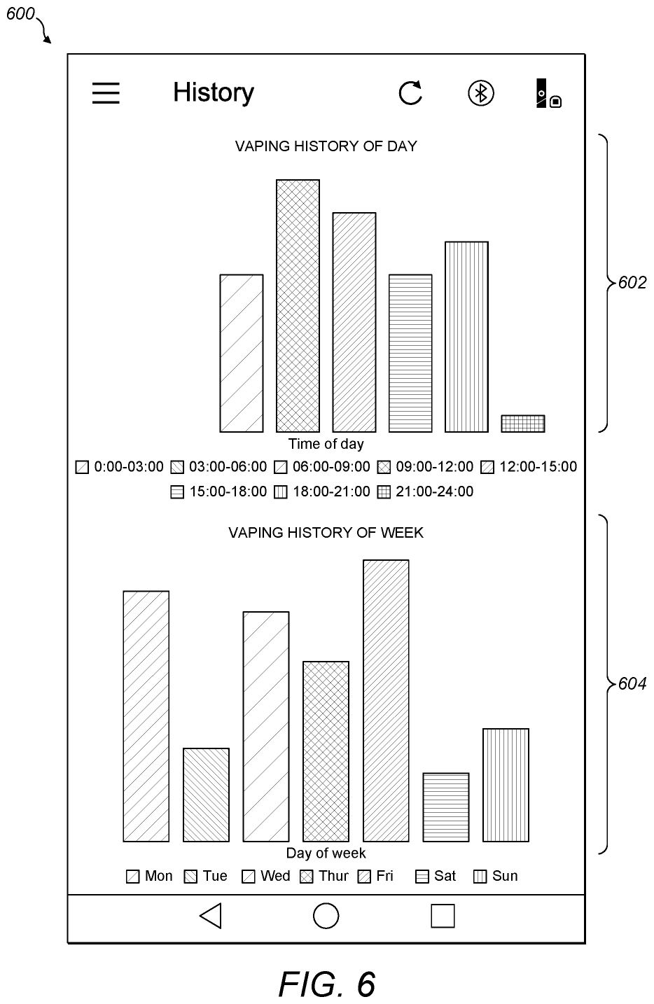

FIG. 6 shows an exemplary graphical user interface 600 that presents information to a consumer derived from the event information received by the communication interface 522 from the aerosol generation device 520. The timestamping allows for a time and date to be assigned to the puffs. The graphical interface 600 displays a consumer's vaping history. In the example, this is displayed in an hourly arrangement 602 and daily arrangement 604 and is determined based upon the timestamped event information.

In low power mode the internal clock of the aerosol generation device 520 is switched off or suspended (i.e. set to a “not running” state). In effect, entering the low power state holds the internal clock at the time at which it was suspended. When the waking trigger is detected, and the device exits the low power mode, the internal clock will start running again from the time at which it was switched off (or a default time such as 00:00:00), this is considered an initial internal time point (T_{INITIAL_INTERNAL}). As such, the time of the internal clock (i.e. the internal time) will not match the real-world external time.

When the aerosol generation device 520 connects to the external electronic device 524 by the communication interface 522, the controller determines the external device time (that is, the clock time of the external electronic device 524) and the internal clock is updated (or synchronised) to this external clock time (i.e. the external time) using the clock time of the external device 524. In an example, the application writes to a DeviceClock characteristic in the device information Bluetooth service. In this way, the new, ‘out of the box’ aerosol generation device 520 can have its internal clock updated from the internal time to the external time when it is first connected to the external electronic device 524.

If a user uses a new ‘out of the box’ aerosol generation device 520 before connecting to the external device 524, i.e. an aerosol generation device 520 that has exited the low power mode but the internal clock has not yet been updated to the external time, the aerosol generation device 520 will record timestamps for the event data relative to the initial internal time point. Such internal timestamps, T_{INTERNAL_STAMP}, use the internal time, based upon the elapsed time from the initial internal time point.

When synchronising with the external electronic device clock, the controller determines the activation time of the aerosol generation device 520 as the point in time at which the aerosol generation device 520 exited the low power mode based upon the absolute external time, rather than the relative internal time. The activation time, T_{ACTIVATION}, is calculated as the difference between the present external time, T_{PRESENT_EXTERNAL}, (i.e. the time of the external electronic device during synchronisation) and the present internal time, T_{PRESENT_INTERNAL}, (i.e. the time of the internal clock relative to the initial internal time when the aerosol generation device exited the low power mode):

T_{ACTIVATION}=T_{PRESENT_EXTERNAL}−T_{PRESENT_INTERNAL}

To facilitate the simple subtraction and addition of clock times, the clock times can be stored as Epoch times.

The controller updates each of the internal timestamps, T_{INTNERAL_STAMP}, to external timestamps (that is timestamps according to the external time), T_{EXTERNAL_STAMP}, using the activation time, T_{ACTIVATION}, and the initial internal time, T_{INITIAL_INTERNAL}:

T_{EXTERNAL_STAMP}=(T_{INTERNAL_STAMP}−T_{INITIAL_INTERNAL})+T_{ACTIVATION}

Alternatively, the difference between the activation time, T_{ACTIVATION}, and the initial internal time, T_{INITIAL_INTERNAL}, can be added to each internal timestamp, by the controller, to update the internal timestamps to external timestamps.

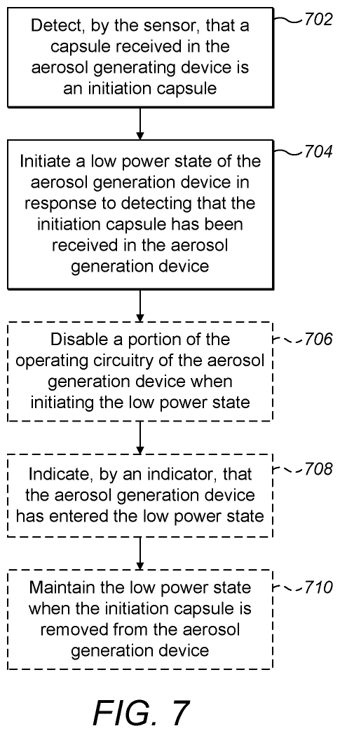

FIG. 7 shows an exemplary flow diagram of the operating steps executed by the controller of the aerosol generation device relating to initiating and exiting the previously described low power mode.

At step 702 the controller detects by the sensor, that a capsule received in the aerosol generation device is an initiation capsule.

At step 704 the controller initiates the low power state for the aerosol generation deice in response to detecting that the initiation capsule has been received in the aerosol generation device.

At step 706 the controller disables a portion of the operating electronics of the aerosol generation device when initiating the low power state.

Optionally, at step 708 the controller indicates by an indicator, that the aerosol generation device has entered the low power state.

Optionally, at step 710 the controller maintains the low power state when the initiation capsule is removed from the aerosol generation device.

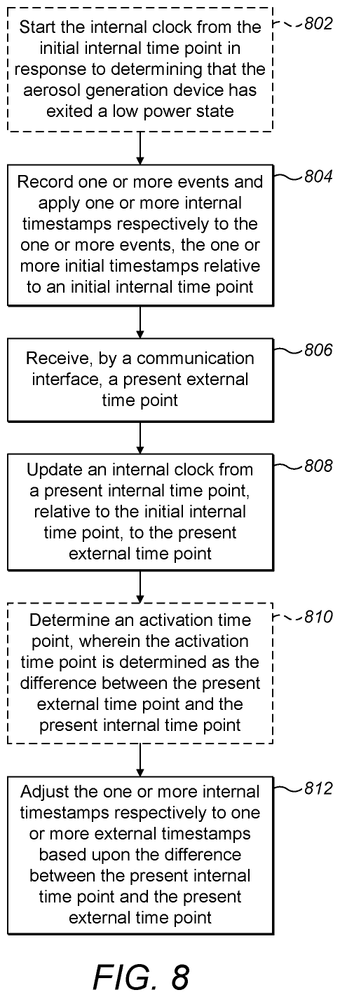

FIG. 8 shows an exemplary flow diagram of the operating steps executed by the controller of the aerosol generation device relating to the previously described timestamp updating process.

Optionally, at step 802 the controller starts the internal clock from the initial internal time point in response to determining that the aerosol generation device to exited a low power state.

At step 804 the controller records one or more events and applies one or more internal timestamps respectively to the one or more events, the one or more initial timestamps relative to an initial internal time point.

At step 806 the controller receives, by a communication interface, a present external time point.

At step 808 the controller updates the internal clock from a present internal time point, relative to the initial internal time point, to the present external time point.

Optionally, at step 810 the controller determines an activation time point, wherein the activation time point is determined as the difference between the present external time point and the present internal time point.

At step 812 the controller adjusts the one or more internal timestamps respectively to one or more external timestamps based upon the difference between the present internal time point and the present external time point.

In addition to the power saving provided by the low power mode for shipping and storage, further power saving can be achieved between uses by the consumer by entering the aerosol generation device into a standby mode. Between uses, when the user is not using the aerosol generation device the lid or cover 224, 324 can be arranged in the closed position. By way of a suitable sensor, such as that previously described with reference to the waking trigger, the controller can determine that the lid or cover 224, 324 is in the closed position. When determining that the lid or cover 224, 324 is in a closed position, the controller can cause the aerosol generation device to enter a standby mode to conserve power. Alternatively or additionally, after determining that the lid or cover 224, 324 has been left in an open position for an amount of time exceeding a preset threshold, the controller can cause the aerosol generation device to enter the standby mode. The preset threshold can be configured in the application at the external electronic device, and instructed to the aerosol generation device using the communication interface.

The standby mode involves suspending at least some of the sub-circuits of the operating electronics that are not essential to the operation of the aerosol generation device when the aerosol generation device is not in use. This preserves battery charge. In operation, a consumer opens the lid or cover 224, 324 to insert the aerosol generating material. The controller determines that the lid or cover 224, 324 has been opened and causes the aerosol generation device to exit the standby mode by powering-on the suspended sub-circuits. In more detail, in the standby mode, the output of the supply switching sub-circuit 404 is switched off, thereby switching off the device temperature cut-out 410, resistance measurement 418, and heater driver 412 sub-circuits.

The processing steps described herein carried out by the main control unit, or controller, may be stored in a non-transitory computer-readable medium, or storage, associated with the main control unit. A computer-readable medium can include non-volatile media and volatile media. Volatile media can include semiconductor memories and dynamic memories, amongst others. Non-volatile media can include optical disks and magnetic disks, amongst others.

It will be readily understood to the skilled person that the preceding embodiments in the foregoing description are not limiting; features of each embodiment may be incorporated into the other embodiments as appropriate.

1. An aerosol generation device comprising:
an internal clock;
a communication interface; and
a controller configured to:
record one or more events and apply one or more internal timestamps respectively to the one or more events, the one or more initial timestamps relative to an initial internal time point;
receive, by the communication interface, a present external time point;
determine an activation time point, wherein the activation time point is determined as a difference between the present external time point and a present internal time point;
update the internal clock from the present internal time point, relative to the initial internal time point, to the present external time point; and
adjust the one or more internal timestamps respectively to one or more external timestamps based upon the difference between the present internal time point and the present external time point;
wherein the controller is configured to adjust a first internal timestamp of the one or more internal timestamps respectively to a first external timestamp of the one or more external timestamps by:
determining a difference between the first internal timestamp and the initial internal time point; and
adding the difference between the first internal time-stamp and the initial internal time point to the activation time point.

2. The aerosol generation device of claim 1, wherein the controller is further configured to start the internal clock from the initial internal time point in response to determining that the aerosol generation device has exited a low power state.

3. The aerosol generation device of claim 1, wherein the controller is configured to receive the present external time point, by the communication interface, from an application executed on an electronic device in communication with the aerosol generation device.

4. The aerosol generation device of claim 3, wherein the controller is configured to update the internal clock to the present external time point when the aerosol generation device first connects to the electronic device.

5. The aerosol generation device of claim 3, wherein the present external time point comprises a present clock time of the electronic device.

6. The aerosol generation device of claim 3, wherein the communication interface is a Bluetooth interface, and the controller is configured to receive the present external time point by a Bluetooth connection to the electronic device using the Bluetooth interface.

7. The aerosol generation device of claim 1, wherein the controller is configured to update the internal clock by writing the present external time point to the internal clock of the aerosol generation device.

8. The aerosol generation device of claim 2, wherein the low power state is a power state in which a portion of operating circuitry used by the aerosol generation device in a fully operational state is disabled.

9. The aerosol generation device of claim 2, wherein the internal clock of the aerosol generation device is disabled prior to exiting the low power state.

10. The aerosol generation device of claim 2, wherein the low power state is configured for shipping and/or storage of the aerosol generation device.

11. The aerosol generation device of claim 1, wherein the initial internal time point, the present internal time point and the one or more internal timestamps are epoch times relative to a reference point internal to the aerosol generation device, and the present external time point and the one or more external timestamps are epoch times relative to a reference point external to the aerosol generation device.

12. The aerosol generation device of claim 1, wherein the one or more events comprise data relating to an inhalation on the aerosol generation device.

13. An aerosol generation device internal clock adjustment method, the method comprising:
recording one or more events and applying one or more internal timestamps respectively to the one or more events, the one or more initial timestamps relative to an initial internal time point;
receiving a present external time point;
determining an activation time point, wherein the activation time point is determined as a difference between the present external time point and a present internal time point;
updating an internal clock of an aerosol generation device from the present internal time point, relative to the initial internal time point, to the present external time point; and
adjusting the one or more internal timestamps respectively to one or more external timestamps based upon the difference between the present internal time point and the present external time point;
wherein adjusting a first internal timestamp of the one or more internal timestamps respectively to a first external timestamp of the one or more external timestamps comprises:
determining a difference between the first internal timestamp and the initial internal time point; and
adding the difference between the first internal time-stamp and the initial internal time point to the activation time point.

14. A non-transitory computer-readable medium storing instructions that, when executed by one or more processors, cause the one or more processors to carry out the steps of:
recording one or more events and applying one or more internal timestamps respectively to the one or more events, the one or more initial timestamps relative to an initial internal time point;
receiving a present external time point;
determining an activation time point, wherein the activation time point is determined as a difference between the present external time point and a present internal time point;
updating an internal clock of an aerosol generation device from the present internal time point, relative to the initial internal time point, to the present external time point; and
adjusting the one or more internal timestamps respectively to one or more external timestamps based upon the difference between the present internal time point and the present external time point;
wherein adjusting a first internal timestamp of the one or more internal timestamps respectively to a first external timestamp of the one or more external timestamps comprises:
determining a difference between the first internal timestamp and the initial internal time point; and
adding the difference between the first internal time-stamp and the initial internal time point to the activation time point.
